(19) United States
(12) Patent Application Publication
(10) Pub. No.: US 2008/0234172 A1
(43) Pub. Date: Sep. 25, 2008

(54) Substrate Care Product

(75) Inventors: Thomas McGee, Nyack, NY (US); Richard P. Sgaramela, Hoboken, NJ (US)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/720,226

(22) PCT Filed: Nov. 28, 2005
(86) PCT No.: PCT/CH05/00707

§ 371 Date: Aug. 1, 2007

Related U.S. Application Data
(60) Provisional application No. 60/631,553, filed on Nov. 29, 2004.

(51) Int. Cl. C11D 17/00; A61K 8/11; C11D 3/50

(52) U.S. Cl. 510/519; 512/4; 428/402.2

(57) ABSTRACT

A substrate care product, such as a fabric conditioner, that provides to the substrate a primary benefit (such as fabric conditioning) and at least one secondary benefit thereto (such as fragrance), the secondary benefit being provided by at least one encapsulated volatile substance that is encapsulated in a capsule having a hydrogel shell and an oily core, the volatile substance having been loaded to blank capsules in the presence of water. The product is storage-stable, and the secondary benefit especially long-lasting.

This invention relates to encapsulated volatile materials, particularly fragrances, methods of making the same, and compositions incorporating these encapsulated volatile materials especially in substrate care products.

By “substrate care products” is meant compositions and products that are used to treat, condition, and fragrance substrates. Substrate care products do not include products that are designed to clean a substrate, for example laundry detergents, shampoos, shower gels, and so forth. Substrate care products do include, inter alia, such products as fabric conditioners, tumble dryer sheets, deodorants, fabric sprays and carpet fragrancers.

In addition to the provision of a primary benefit (e.g. conditioning), such substrate care products may also be used to provide at least one secondary benefit. A commonly-desired secondary benefit is the addition of fragrance to a substrate to which the product is applied. The remainder of this description will refer specifically to fragrance, but the invention is not restricted to fragrance but is applicable to any volatile substance that provides additional benefits.

Ideally, not only should such a substrate care products deliver a fragrance successfully, but that fragrance should also be long-lasting. This in turn implies that:
1. There should be an acceptably low loss of fragrance prior to use, so that as much of the original fragrance loading as possible is present when delivery occurs;
2. Once deposited on the substrate, the fragrance should be perceptible for as long as possible.

With regard firstly to No.2, one way is to choose fragrances with the inherent property of endurance. Examples of this approach is to be found in U.S. Pat. No. 5,652,206 and No. 5,833,999, which discloses that, for enduring fragrances, a fragrance must be composed of particular types of fragrance materials, namely, a small number of specifically-named materials, plus at least 70% of materials having a ClogP of greater than 3.0 and a boiling point of greater than 250° C. (ClogP, the calculated logarithm of the octanol/water partition coefficient, which gives an indication of hydrophilicity, is a well-known parameter and may be calculated for any given molecule from a knowledge of the structure of that molecule. There are a number of commercially-available computer programs that can do this, for example, ACD Software, ACD/logP calculator version 4.0, Advanced Chemistry Development, Toronto, Ontario Canada.) While undoubtedly providing enduring fragrance, this method is unduly restrictive with respect to the fragrances that can be used—a great many desirable fragrance materials fall outside these limits.

A potential method of providing quantities of fragrance to a substrate is by encapsulation, in which the fragrance is provided in capsules, which release the fragrance only under the desired conditions. Many such encapsulation methods are known to the art, and many are suitable for use with fragrances. However, these systems have drawbacks. Their manufacture can be expensive and they can be unstable on storage. Alternatively, they may not release fragrance when required, or until some stimulus, such as pressure, heat or water is applied. When the fragrance is released on to the treated substrate, it may happen all at once, rather than as a desirable controlled release of fragrance over time.

It has now been found that the application of a particular microcapsular technology for use in substrate care products can substantially or even completely overcome the abovementioned problems of the art. In one aspect, the present invention provides a substrate care product comprising at least one encapsulated volatile substance that is encapsulated in a capsule having a hydrogel shell and an oily core, the volatile substance having been loaded into blank capsules in the presence of water. In another aspect of the invention there is provided a substrate care product adapted to be applied to a substrate to provide a primary benefit and at least one secondary benefit thereto, the secondary benefit being provided by at least one encapsulated volatile substance that is encapsulated in a capsule having a hydrogel shell and an oily core, the volatile substance having been loaded into blank capsules in the presence of water. In further aspects the invention additionally provides a method of providing to a substrate a primary benefit and at least one secondary benefit, comprising treating the substrate with a substrate care product comprising at least one encapsulated volatile substance as hereinabove described.

It has been surprisingly discovered that relatively low cost microcapsules, prepared by a process as herein described, are very stable in relatively low surface active products, and they release their at least one encapsulated volatile substance, desirably a fragrance, on a treated substrate without the need of pressure, temperature or water. Additionally the volatile raw materials or accords that would easily evaporate during the drying stage can be retained and released in over an extended period of time. Further details of the microcapsules will be provided hereinunder, but relevant processes involved are fully described and exemplified in U.S. Pat. Nos. 6,045,835 and 6,106,875, the contents whereof are incorporated herein by reference.

The at least one encapsulated volatile substance is desirably a fragrance which is contained in capsules whose shell is formed from a suitable protein, for example gelatine, albumin, casein, lacto-globulin and alginate. These, especially gelatine, allow the achievement of a long-lasting effect on treated dry fabric. Any type of gelatine employed in the manufacture of capsules for use in food, consumer product and medicinal fields may be employed in the present invention. Alternatively, the shell may be formed of gum arabic.

The classes of fragrance materials that can be encapsulated and released from the microcapsules of this invention are not limited to any chemical structure. The principal classes of fragrance materials are given in S. Arctander, Perfume and Flavor Chemicals (Montclair, N.J., 1969). The fragrance can include solvents known in the art.

It has been found that a significant quantity of the fragrance can be encapsulated. U.S. Pat. Nos. 6,045,835 and 6,106,875 teach that a flavor or fragrance compound that is hydrophilic may be less readily contained in the oil core of the particle. However, it has been found that wide range of hydrophilic compounds may be incorporated if present as part of a mixture. Materials with a ClogP as low as around 1.0 can be successfully incorporated.

In creating a fragrance for encapsulation according to this invention, the rate of diffusion of the fragrance from the microcapsule may be indicated by the average vapor pressure of the mixture of the fragrance materials, which may be calculated using the vapor pressure of the individual components of the volatile mixture as follows:

average vapor pressure=Σ(aᵢ×VPᵢ)/n

where aᵢ is the percentage of ingredient i mixture,
and VPᵢ is the vapor pressure in mmHg@25° C. of the ingredient i

The vapor pressure can either be measured or calculated from the molecular structure using one of the commercially-available software programs, such as ACD Software ACD/Boiling Point calculator version 4.0, Chemistry Development, Toronto, Ontario Canada. Solvents that are known in the art can be part of the fragrance composition and their vapor pressure is taken into account.

The desired level and length of perception of a fragrance (or other volatile material) will depend on the end-use of the products in which the fragrance is incorporated. For example, in the case of a personal deodorant, it is desirable for fragrance perception to be highest over 24 hours after application. On the other hand, for a fabric conditioner, perception over about 14 days is desirable. The skilled person using the ordinary skill of the art and with only routine experimentation will readily be able to provide the necessary perception.

In order to maximize the intensity and hence perception of this fragrance, more than 20% of the ingredients should have a perception threshold about less than about 300 nanograms per liter, preferably less than 20 nanograms per liter, most preferably less than about 10 nanograms per liter. A variety of techniques are available to determine the perception threshold. See for example, Neuener-Jehle and Etzweiler in Art Science and Technology, editors Lampaski and Muller, Ch.6, 153-212 Elsevier Science Publishers Ltd. 1991.

The fragrance (or other volatile materials) may be incorporated into the microcapsules when they are in a dry form or when they are in a liquid suspension. The fragrance and the microcapsules are mixed until the capsules become dry and free flowing

A preferred embodiment of the invention is that the microcapsules are prepared in a dry form. The fragrance compound is added in the presence of a controlled volume of water or water-alcohol mixture, to a substantially dry microcapsule having a hydrogel shell surrounding an oil core. The compound is transported through the hydrogel shell by aqueous diffusion into the oil core and is retained in the core. The microcapsule having the fragrance compound is retained in the oil core and is then dried.

In certain preferred embodiments of the invention the particle size of the microcapsules are smaller than 2400 microns preferably less than 400 microns and most preferably less than 150 microns.

According to a further inventive embodiment the surface care product is a fabric conditioner with a pH of less that 8.0 which further contains the encapsulated volatile substance, preferably a fragrance. The fabric conditioner may be in a liquid, semi-solid (e.g, paste, gel) or solid form. A ‘conditioner’ is a material or mixture of materials that improve the softness and reduce the static charge of the treated substrate, preferably a fabric, textile, garment, etc. The conditioner material or mixture of materials include compounds can be selected from cationic, nonionic, amphoteric and/or anionic compounds known in the art to impart softness and/or antistatic properties to fabrics, textiles as well as finished articles made from fabrics and textiles.

Any known-art compounds useful as conditioners may be used. Non-limiting examples of such compounds include:

1. Cationic quaternary ammonium salts, such as: acyclic quaternary ammonium salts having at least two C₈₋₃₀, preferably C₁₂₋₂₂ alkyl chains, such as: ditallowdimethyl ammonium chloride, ditallowdimethyl ammonium methylsulfate, di(hydrogenated tallow)dimethyl ammonium methylsulfate, distearyldimethyl ammonium methylsulfate, dicocodimethyl ammonium methylsulfate and the like; Cyclic quaternary ammonium salts of the imidazolinium type such as di(hydrogenated tallow)dimethyl imidazolinium methylsulfate, 1-ethylene-bis(2-tallow-1-methyl) imidazolinium methylsulfate and the like; diamido quaternary ammonium salts such as: methyl-bis(hydrogenated tallow amidoethyl)-2-hydroxyethyl ammonium methyl sulfate, methyl bis(tallowamidoethyl)-2-hydroxypropyl ammonium methylsulfate and the like; Biodegradable quaternary ammonium salts such as N,N-di(tallowoyl-oxy-ethyl)-N,N,-dimethyl ammonium methyl sulfate and N,N-di(tallowoyl-oxy-propyl)-N,N-dimethyl ammonium methyl sulfate. Examples are given in U.S. Pat. Nos. 4,137,180, 4,134,838, 4,767,547 and 4,789,491 and are incorporated by reference herein.

2. Tertiary fatty amines having at least one and preferably two C₈₋₃₀, preferably C₁₂₋₂₂ alkyl chains. Examples include hardened tallow amine and cyclic amines such as 1-(hydrogenated tallow)amidoethyl-2-(hydrogenated tallow) imidazoline. Cyclic amines are described in U.S. Pat. No. 4,806,255, which is incorporated by reference

3. Carboxylic acids having 8 to 30 carbon atoms and one carboxylic group per molecule, the alkyl portion has 8 to 30, preferably 12 to 22 carbon atoms. The alkyl portion may be linear or branched, saturated or unsaturated, with linear saturated alkyl preferred.

4 Anionic Surfactant such water-soluble salts of the higher fatty acids, i.e., “soaps”. These include alkali metal soaps such as the sodium, potassium, ammonium, and alkylolammonium salts of higher fatty acids containing from about 8 to about 24 carbon atoms, and preferably from about 12 to about 18 carbon atoms as cited in U.S. Pat. No. 6,133,226.

5. Fatty acid partial esters of polyhydric alcohols, or anhydrides thereof, wherein the alcohol, or anhydride, contains from 2 to 18, preferably from 2 to 8, carbon atoms, and each fatty acid moiety contains from 12 to 30, preferably from 16 to 20, carbon atoms. Typically, such softeners contain from one to 3, preferably 2 fatty acid groups per molecule. The polyhydric alcohol portion of the ester can be ethylene glycol, glycerol, poly (e.g., di-, tri-, tetra, penta-, and/or hexa-) glycerol, xylitol, sucrose, erythritol, pentarytiritol, sorbitol or sorbitan. Sorbitan esters and polyglycerol monostearate are particularly preferred.

6. Nonionics such as fatty alcohols, ethoxylated fatty alcohols, alkylphenols, ethoxylated alkylphenols, ethoxylated fatty amines, ethoxylated monoglycerides and ethoxylated diglycerides. For example, U.S. Pat. Nos. 4,209,549 and 5,145,595 disclose a ethoxylated nonionics as anti-static ingredients.

7. Oils such as mineral oils, mineral oils, ester oils, sugar ester oils or oily sugar derivatives, natural oils, such as vegetable oils, and mixtures thereof, and polyols such as polyethylene glycol as described in WO 97/44424 and WO98/16538, which are incorporated by reference as if recited in full herein.

8. Silicones, such as aminosilicones such as those disclosed in U.S. Pat. No. 4,891,166 and European Patent No 459 821.

The fabric conditioner compositions according to the present invention are preferably designed for addition to the rinse stage of a wash cycle are typically in a liquid form, but other forms such as semi-solid, or comminuted solids such as in powder or granulate form is also possible. They may be formulated for use in any convenient manner.

A further embodiment of inventive substrate care products are ones in which the composition is a waxy solid composition that has a melting (or softening) point of from 35°-150° C. which in addition to a ‘conditioner’ which improves the softness and reduce the static charge of the treated substrate further contains the encapsulated volatile substance, especially preferably a fragrance, which waxy solid composition is provided on or in a carrier that is placed in a tumble dryer. The conditioner itself may be the waxy solid composition, or form part of the waxy solid composition. The term “carrier” covers any means, article or composition for delivering the conditioner and capsules of the invention to the fabric in the tumble dryer. Preferably, however, the carrier is a flexible sheet, such as of woven or non-woven fabric made from synthetic or natural materials, paper sheet or porous sponge that has the conditioner containing the capsules of the invention coated thereon. Alternately the carrier may be a container having one or more apertures into which the a conditioner containing capsules of the invention is provided; the one or more apertures to allow for the conditioner and capsules to pass out of the container in the tumble dryer and come into contact with the fabric.

When the carrier is a sheet, the capsules can be incorporated into the waxy solid composition in a coating bath maintained at around 40° C., preferably with stirring to maintain the particles suspended. The dryer sheet carrier is coated by being passed over an applicator roll. In its passage over this roll, the sheet is coated with a thin, uniform layer of molten composition. The carrier is passed over a cooling roll to solidify the composition. The roll may then be cut into sheets and packed. Alternative ways of incorporating the microcapsules into the waxy solid composition may be used, for example, by applying the waxy solid composition minus the fragrance-containing microcapsules to the carrier and then spraying the microcapsules on to the carrier between the coating roller and the cooling roller.

Another embodiment is a tumble dryer sheet in which in addition to the fragrance contained in the capsules the fragrance is added closer to the step of packaging, i.e. after application of other ingredients in the coating pan.

The capsules of this invention can also be incorporated into a carrier that is formulated with subliming solids along with the conditioner such that in the tumble dryer the conditioner and the capsules are transferred to the fabric without the need to have to retrieve a substrate. By way of non-limiting example such a subliming solid includes adamantane and derivatives thereof.

Alternatively, substrate care products of this invention may be applied to a fabric as a sprayable composition either before or during drying. The spray may be applied in a tumble dryer. Suitable dispensers for the spray include aerosols and spray delivery devices, with apertures or nozzles which permit the passage of the microcapsules.

A preferred embodiment is one in which, in addition to the fragrance contained in the capsules, fragrance is also added in a non-encapsulated form to the substrate care products, so that the product has a desirable scent, and when added to the final rinse water, this desirable scent is imparted to the solution and to the damp substrate.

The compositions of substrate care products of the present invention may include other optional components conventionally used in textile treatment compositions, for example, colorants, preservatives, optical brighteners, opacifiers, surfactants, stabilizers, viscosity modifiers, anti-shrinkage agents, anti-wrinkle agents, fabric crisping agents, anti-spotting agents, germicides, fungicides, anti-corrosion agents, anti-foam agents, enzymes such as celluloses, proteases, and the like, dye transfer inhibitors, chlorine scavengers, soil release agents, non-aqueous solvents, hydrotropes, antifoaming agents, anti-redeposition agents, anti-oxidants, ultra violet absorbers, heavy metal sequestrants, dye fixatives, anti-corrosion agents, drape imparting agents, and ironing aids. This list is not intended to be exhaustive and other optional components not specifically elucidated here but known in the respective art may also be used.

Yet further aspects of the invention are substrate care products meant for fragrancing and deodorizing substrates, such as carpets, upholstery, fabric and drapes. The formulation of such substrate care products can be applied in several ways, such as sprays and foams. They have a relatively low level of surfactant, generally less than about 15% wt. of one or more surfactants. For example, U.S. Pat. No. 6,524,494 discloses a fabric refreshing composition, based on water soluble silicones a fragrance composition; and an aqueous carrier comprising an emulsifier, to fragrance and reduce wrinkles of treated fabrics. Many other routes to achieving this are in the art, for example, U.S. Pat. No. 6,503,413 discloses a spray to treat substrates, especially fabrics, to fragrance and reduce malodor and the appearance of wrinkles. These products can also be used as ironing aids. Another type of surface care product is exemplified in U.S. Pat. Nos. 6,482,783 and 6,315,949 which discloses fabric freshening agent, such as, fragrance, in the form of an aerosol foam, to show where the product has been applied, eliminating the need to vacuum without leaving behind residues. The inclusion of the capsules according to the invention within the formulation of these products significantly enhances the fragrance longevity in these types of substrate care products. These substrate care products may include additional optional ingredients, e.g., antifoaming agents to assist in the quick breakup foam, anti-resoiling additives, organic solvents, odor absorbers, preservatives, antioxidants, antimicrobials, ultra violet absorbers, heavy metal sequestrants, and the like. This list in not meant to be exhaustive. In particularly preferred embodiments of substrate care products meant for fragrancing and deodorizing substrates are those which, in addition to the fragrance contained in the capsules, additional non-encapsulated fragrance is added to the substrate fragrancing composition, so that the resultant product has a desirable scent.

The capsules of this invention can also be used in personal deodorant products, which demonstrate a further form of substrate care products. These products essentially have low surface activity. The surfactants present in such personal deodorant products are primarily included for emulsification of the fragrance. Exemplary known-art formulations are too numerous to exemplify, although they range from simple opaque stick products produced by gelling water with either sodium stearate or sodium palmitate through translucent and transparent sticks produced by selecting materials with the correct refractive index. Liquids and soft solid compositions are also well known in the art of personal deodorant products. These products are primarily aimed at reducing body odor. The capsules of this invention provide long lasting fragrance that is particularly useful in providing malodor coverage and fragrancing over extended periods. Such personal deodorant products can further contain optional ingredients such as antiperspirants, antimicrobials, enzyme inhibitors, odor absorbers, odor neutralizers, dyes, colorants, anti-stick agents, skin feel ingredients, anti-inflammatory ingredients, cooling ingredients, and the like. This list is not meant to be exhaustive.

A preferred embodiment is one in which, in addition to the fragrance contained in the capsules, fragrance is added to the personal deodorant products composition, so that the product has a desirable scent.

The following non-limiting examples illustrate the compositions, method of making, and methods of using the present invention described in this application.

### EXAMPLE 1

The following fragrances were constructed to explore the relationship of hydrophilicity on the ease of manufacturing of the encapsulates.

(i) Fragrance 1: Low Average ClogP (Average 1.8)

| Ingredient | ClogP | %(w/w) |
|---|---|---|
| GUAIACOL | 1.19 | 18.87 |
| LINALOOL OXIDE | 1.56 | 18.87 |
| CAMPHOR SYNTHETIC TECHNICAL | 1.65 | 14.15 |
| ACETOPHENONE EXTRA | 1.66 | 14.15 |
| PHENYL ACETALDEHYDE | 1.78 | 14.15 |
| METHYL BENZOATE | 2.2 | 14.15 |
| ALDEHYDE C9 | 3.56 | 0.94 |
| HEXYL BUTYRATE | 3.9 | 0.94 |
| CYDRANE | 4.24 | 0.94 |
| TETRAHYDRO LINALYL ACETATE | 4.41 | 0.94 |
| TERPINENE, GAMMA | 4.36 | 0.94 |
| CARYOPHYLLENE | 6.78 | 0.94 |

(ii) Fragrance 2: High ClogP formulation (Average 4.5)

| Ingredient | ClogP | %(w/w) |
|---|---|---|
| GUAIACOL | 1.19 | 0.9 |
| LINALOOL OXIDE | 1.56 | 0.9 |
| CAMPHOR SYNTHETIC TECHNICAL | 1.65 | 0.9 |
| ACETOPHENONE EXTRA | 1.66 | 0.9 |
| PHENYL ACETALDEHYDE | 1.78 | 0.9 |
| METHYL BENZOATE | 2.2 | 0.9 |
| ALDEHYDE C9 | 3.56 | 13.51 |
| HEXYL BUTYRATE | 3.9 | 13.51 |
| CYDRANE | 4.24 | 13.51 |
| TETRAHYDRO LINALYL ACETATE | 4.41 | 18.02 |
| TERPINENE, GAMMA | 4.36 | 18.02 |
| CARYOPHYLLENE | 6.78 | 18.02 |

These fragrances were each loaded at 20% into gelatin microcapsules and gum arabic capsules obtained from Givaudan Flavors, Cincinnati, USA, according to the procedure hereinunder described. The code numbers are those under which the capsules are available.

Procedure:

1. Mix fragrance, alcohol and water (liquid phase) in the following proportions:

| | %(w/w) |
|---|---|
| capsules | 65 |
| Fragrance | 20 |
| Alcohol | 10 |
| Water | 5 |

2. Stir vigorously until an emulsion is formed.

3. Pour the liquid phase emulsion over the caps in a bottle and homogenize the mixture.

4. Cap the bottle and leave it tumbling until the powder is dry and free flowing.

The following observations were made:

| | Time to form |
|---|---|
| Experiment | dry powder |
| Fragrance 1- Gum Arabic (AG: 22-173-00) Capsules | 10 minutes |
| Fragrance 2- Gum Arabic (AG: 22-173-00) Capsules | 45 minutes |
| Fragrance 1- Gelatin (SBB: 96655855) Capsules | 10 minutes |
| Fragrance 2- Gelatin (SBB: 96655855) Capsules | 45 minutes |

Both the very low average ClogP and very high average ClogP could be made into dry free flowing loaded capsule. Fragrance 2, which had the higher average ClogP fragrance, took longer to form capsules than did Fragrance 1 which had the lower average ClogP.

### EXAMPLE 2

The following fruity fragrance accord was created:

| Ingredient | %(w/w) | ClogP |
|---|---|---|
| Amyl Acetate | 5 | 2.1 |
| Ethyl caproate | 10 | 2.8 |
| Hexyl Acetate | 10 | 2.8 |
| Allyl Caproate | 10 | 3.1 |
| Methyl-4,7-Octadienoate | 5 | 1.6 |
| Allyl Heptoate | 15 | 3.7 |
| Allyl Cyclohexyl Proprionate | 25 | 4.1 |
| Acetate PA | 10 | 2.3 |
| 2,4-Dimethyl-2-(1,1,4,4- | 10 | 5.7 |
| teramethyltetralin-6-yl)-1,3-dioxolane | | |
| Average ClogP | | 3.8 |

The fragrance was loaded at 20% (w/w/) into the dry gum arabic FB-D: 22-173-00 capsules and gelatin FB-D: 96655855 microcapsules as above.

The gelatin MCT 2× FB-L:22-166-00 and gelatin 50μ FB-L: 96404445 capsules were obtained suspended in the liquid phase. 20% (w/w) of the fragrance was added slowly to the capsules in liquid in a bottle. The bottle was capped and tumbled for 3 hours. The capsules were dry and free flowing at the end of the tumbling.

5 cm square pads placed in Petri dishes were dosed with 40 mg of the fragrance accord and 200 mg (40 mg neat oil equivalent of the respective capsules loaded at 20%; the quantity of fragrance delivered by the capsules was 40 mg). The samples were allowed to dry overnight. Headspace was collected at day 1, day 3 and day 5 and analyzed using gas chromatography and mass spectrometry. The following results were obtained:

| | Headspace Nanograms per Liter | | |
|---|---|---|---|
| | Day1 | Day 3 | Day 5 |
| Gum arabic FB-D: 22-173-00 | 202 | 183 | 103 |
| Gelatin MCT 2x FB-L: 22-166-00 | 177 | 155 | 97 |
| Gelatin 50μ FB-L: 96404445 | 115 | 102 | 76 |
| Gelatin FB-D: 96655855 | 344 | 422 | 488 |
| Free Scent Accord | 37 | 6 | 0 |

All beads provide a greater fragrance in the headspace than the scent accord alone.

### EXAMPLE 3

The fruity fragrance described in Example 2 was loaded at 20% (w/w) in the Gelatin FB-D: 96655855 capsules, using the method outlined in Example 1.

A pilot plant used for making tumble dryer sheets (TDS) consists of a coating pan and an application roller. A sheet weighing 1 g was prepared by passing it through one of two coating baths as hereinunder described and then through the roller. The sheet was weighed and re-passed through the roller until 1.4 grams of conditioner had been applied

The composition of the basic coating bath was as follows:

| Ingredients | %(w/w) |
|---|---|
| Potassium Stearate | 25 |
| Stearic Acid | 46 |
| Linear ethoxylated fatty acid (Neodol ™ 25-9) | 29 |

To each bath was added the following:

| Product | Free Fruity fragrance | Capsule containing the fruity fragrance |
|---|---|---|
| A | 1.05% | — |
| B | — | 5.25% @ 20% loading* |

*Amount of fragrance equivalent to 1.5% the free Fruity fragrance.

Half the sheets thus prepared were placed in a 40° C. storage oven and left for 12 days to check stability.

Terry toweling test pieces were washed in Purex™ Fragrance Free Detergent liquid. The product concentration of the Purex™ in a Whirlpool™ Twin automatic washer/dryer washing machine was 0.2% and the cloth to liquor ratio of was 1:20. After the toweling was rinsed and spun dry it was placed in the dryer on the hot cycle for 50 minutes using the respective TDS described above. The above wash and dry procedure was repeated using the tumble dryer sheets stored at 40° C. for 12 days.

The headspace of the towels was taken after 3 days the following result was obtained:

| TDS | Headspace after 3 days | |
|---|---|---|
| Neat fragrance Accord | 1.5 | nanograms/liter |
| Capsule | 213.8 | nanograms/liter |
| Capsule Stored 12 days | 239.3 | nanograms/liter |

### EXAMPLE 4

TDSs were prepared from the conditioner bases given below, using the method described in Example 3, containing 5.25% Gelatin FB-D: 96655855 capsules with 20% of the fruity fragrance accord as per Example 2: Towels were washed and dried as per example 3.

Soap/nonionic base, same formulation as Example 3

Cationic Base as follows:

| | % |
|---|---|
| Ingredient | (w/w) |
| Varisoft ™ 136-100 (1) | 90 |
| Ethanol 39C | 5 |
| Water Distilled | 5 |

Varisoft = di-C₁₄-C₁₈ alkyldimethyl quaternary ammonium methyl sulfate

The results on dry towels were as follows:

| | Initial | Day 3 | Day 7 |
|---|---|---|---|
| Soap/Non-ionic | 14.1 | 78.5 | 136.3 |
| | nanograms/liter | nanograms/liter | nanograms/liter |
| Cationic | 14.7 | 82.7 | 142.8 |
| | nanograms/liter | nanograms/liter | nanograms/liter |

This shows that the invention is independent of the base used.

### EXAMPLE 5

The Gelatin FB-D: 96655855 capsules from Example 2 were incorporated into commercial unfragranced low active conditioner base at 5.25% (1.05% Fruity fragrance). A control was prepared by incorporating 1.05% free Fruity fragrance from Example 2 into the dilute fabric conditioner.

Terry toweling test pieces were washed in 0.2% Purex™ fragrance-free detergent liquid in a Terg-O-Tometer laboratory washing machine simulator at a cloth to liquor ratio of 1:20 for 10 minutes. The excess wash liquor was removed and the towels rinsed once in water. A final rinse was carried out in 0.1% of the respective fabric conditioners. The towels were tumble dried and then the headspace collected after 3 days was analyzed by GC/MS. The results are shown below:

| | Headspace after 3 days |
|---|---|
| 1.05% Fruity fragrance | 3.71 ng/l |
| 5.25% Gelatin FB-D: 96655855 capsules @ 20% | 17.5 ng/l |
| Fruity fragrance | |

The capsules are clearly giving much more fragrance on the dry fabric after 3 days. The conditioner was stored for one week at 40° C. to validate storage stability.

The above wash and rinse was repeated and the headspace data is shown below:

| | Headspace after 3 days |
|---|---|
| 5.25% Gelatin FB-D: 96655855 capsules @ 20% | 36.7 ng/l |
| Fruity fragrance - Aged 40° C. 1 week | |

The performance of the aged sample is at least as good as the freshly made sample.

### EXAMPLE 6

Two fragrance accords, a pineapple accord and a floral woody accord, both having an average ClogP of 2.8, were created.

These were loaded, respectively, at 20% in the Gelatin FB-D: 96655855 capsules using the method described in Example 1.

These were incorporated into a commercial unfragranced low active conditioner base in the following way.

| Product | Conditioner | Neat Accord | | Capsules | |
|---|---|---|---|---|---|
| A | 99.00% | 1% | Pineapple | | |
| B | 99.00% | 0.7% | Pineapple | 0.3% | Pineapple |
| C | 99.00% | 1% | Floral woody | | |
| D | 99.00% | 0.7% | Floral woody | 0.3% | Floral woody |

Terry toweling test pieces were rinsed in 0.2% of the conditioner at 1:20 cloth to liquor ratio and the towels were line dried overnight.

The dry towels were submitted to a trained panel, to establish whether the capsules provided a higher intensity fragrance than the free fragrance alone. The Label Magnitude Scale (LMS) was used to evaluate the odor intensity. (Barry G. Green, Gregory S. Shaffer, Magdalena, M. Gilmore, Chemical Senses. Vol. 18, pp 683-702, 1993. Barry G. Green, Pamela Dalton, Beverly Cowart, Greg Shaffer, Krystyna Ranking, Chemical Senses. Vol. 21, pp 323-334, 1996)

| | Intensity |
|---|---|
| Conditioner | Rating |
| A | 1.9 |
| B | 3.9 |
| C | 1.9 |
| D | 3 |

The capsules provide a significantly higher intensity of the dry terry toweling.

### EXAMPLE 7

Sublimable tablets were made using the Fruity fragrance of Example 2 and capsules containing the Fruity fragrance described in Example 2 loaded at 20% (w/w) in the Gelatin FB-D: 9655855 capsules.

| Ingredient | A Wt % | B Wt % |
|---|---|---|
| Adamantane | 18.3 | 18.3 |
| Varisoft DS 150 | 60.0 | 60.0 |
| Fruity Fragrance | 3.4 | 2.4 |
| Gelatin FB-D: 96655855 capsules @ 20% | — | 5.0 |
| Fruity fragrance | | |

Terry toweling test pieces were washed as per Example 2, and after spinning dry the towels were placed in a dryer and the respective tablet added. The towels were dried on the hot cycle.

The towels were stored for 7 days. A panel of 5 experts smelled the intensity, and all rated the intensity of the towels that had been dried with the tablet containing the capsules as significantly more intense that the ones dried with the tablet containing the free fragrance. This shows that the capsules delivered significantly longer lasting fragrance intensity than free fragrance alone.

### EXAMPLE 8

The fragrance and capsules of Example 2 were used to prepare the following deodorant sticks:

| | Stick 1 | Stick 2 |
|---|---|---|
| | % (w/w) | %(w/w) |
| Propylene Glycol | 81.50 | 81.70 |
| Sodium Stearate C-1 | 8.00 | 8.00 |
| 0.25% Triclosan | 0.25 | 0.25 |
| Demineralised water | 5.00 | 5.00 |
| Gelatin FB-D: 96655855 capsules | 5.25 | — |
| Fruity Fragrance | — | 1.05% |

The products were applied to the skin of a panelist by rubbing 5 times on the flex area of each arm. A panel of 5 experts smelled the intensity after 5 hours and all selected the one in which the fragrance had been encapsulated in the capsules of this invention, showing that the capsules delivered a significantly longer lasting fragrance intensity than free fragrance alone

### EXAMPLE 9

The following antiperspirant deodorant sticks were prepared using the fragrance oil and capsule of example 2.

| | |
|---|---|
| Stick 3 | Stick 4 |
| %(w/w) | %(w/w) |

Phase I

| | | | |
|---|---|---|---|
| 1. | Cyclomethicone (Dow Corning 245 Fluid) | 49.75 | 51.95 |
| 2. | Stearyl Alcohol (Crodacol ™ S-95) | 23.00 | 24.00 |
| 3. | Glyceryl Stearate (Arlacel ™ 165) | 1.00 | 1.00 |

Phase II

| | | | |
|---|---|---|---|
| 4. | Aluminum Zirconium Tetrachlorohydrex | 21.00 | 22.00 |
| | Glycine (Rezal ™ 36 GP S.U.F) | | |

Phase III

| | | | |
|---|---|---|---|
| 5. | Gelatin capsules SSB | 5.25 | — |
| 6. | Fragrance Accord | — | 1.05 |

These premixes were combined to form the antiperspirant stick.

The products were applied to the skin of a panelist by rubbing 5 times on the flex area of each arm. A panel of 5 experts smelled the intensity after 5 hours and all selected the one in which the fragrance had been encapsulated in the capsules of this invention, showing that the capsules delivered a significantly longer lasting fragrance intensity than free fragrance alone.

1. A substrate care product adapted to be applied to a substrate to provide a primary benefit and at least one secondary benefit thereto, the secondary benefit being provided by at least one encapsulated volatile substance that is encapsulated in a capsule having a hydrogel shell and an oily core, the volatile substance having been loaded to blank capsules in the presence of water.

2. A substrate care product according to claim 1, in which the secondary benefit provided is fragrance.

3. A substrate care product according to claim 1, in which the shell of the capsule is formed from a material selected from gum Arabic and a protein.

4. A substrate care product according to claim 3, in which the protein is selected from the group consisting of gelatine, albumin, casein, lacto-globulin and alginate.

5. A substrate care product according to claim 2, in which more than 20% of the ingredients of the fragrance have a perception threshold of less than 300 nanograms per liter.

6. A substrate care product according to claim 1, in which the capsules are prepared in dry form.

7. A substrate care product according to claim 1, in which the capsules have a particle size of less than 2400 microns.

8. A substrate care product according to claim 1, which is a fabric conditioner with a pH of less than 8.0.

9. A substrate care product according to claim 1, which is a fabric conditioner, having the form of a waxy solid that has a melting point or softening point of from 35°-150° C., and which, in addition to a conditioner contains the encapsulated volatile substance, which waxy solid composition is provided on or in a carrier adapted to be placed in a tumble dryer.

10. A method of providing to a substrate a primary benefit and at least one secondary benefit, at least one secondary benefit being conferred by a volatile substance, comprising treating the substrate with a substrate care product comprising at least one volatile substance that is encapsulated in a capsule having a hydrogel shell and an oily core, the volatile substance having been loaded to blank capsules in the presence of water.

11. A substrate care product according to claim 3, in which the protein is selected gelatine.

12. A substrate care product according to claim 5, in which more than 20% of the ingredients of the fragrance have a perception threshold of less than 20 nanograms per liter.

13. A substrate care product according to claim 12, in which more than 20% of the ingredients of the fragrance have a perception threshold of less than 10 nanograms per liter.

14. A substrate care product according to claim 7, in which the capsules have a particle size of less than 400 microns.

15. A substrate care product according to claim 14, in which the capsules have a particle size of less than 150 microns.
